(19) United States
(12) Patent Application Publication
(10) Pub. No.: US 2022/0008373 A1
(43) Pub. Date: Jan. 13, 2022

(54) COMPOSITIONS FOR TREATMENT OF VIRAL INFECTIONS

(71) Applicants: Mark Loveless, Portland, OR (US); William Comer, Beaverton, OR (US)

(72) Inventors: Mark Loveless, Portland, OR (US); William Comer, Beaverton, OR (US)

(21) Appl. No.: 17/372,158

(22) Filed: Jul. 9, 2021

Related U.S. Application Data
(60) Provisional application No. 63/050,434, filed on Jul. 10, 2020.

(51) Int. Cl. A61K 31/245; A61K 31/506; A61K 31/496; A61K 9/00; A61P 31/14

(52) U.S. Cl. CPC A61K 31/245; A61K 31/506; A61P 31/14; A61K 9/0073; A61K 31/496

(57) ABSTRACT

The present invention is directed to compositions, methods for preventing, treating and/or curing respiratory infections, such as infections caused by a coronavirus.

### CLAIM OF PRIORITY UNDER 35 U.S.C. § 119

The present Application for Patent claims priority to Provisional Application No. 63/050,434 entitled “COMPOSITIONS FOR TREATMENT OF VIRAL INFECTIONS” filed Jul. 10, 2020, which is hereby expressly incorporated by reference herein.

### BACKGROUND

### Field

The present invention is directed to compositions and methods for preventing, treating and/or curing respiratory infections, such as infections caused by a coronavirus.

### Background

For the treatment of infection or disease by infectious pathogens, most existing therapeutic agents do not have a mechanism of action that blocks the initial interaction of the infectious pathogen or a toxin produced by that pathogen with cells. A small set of therapeutic agents use specific antibodies (hyperimmune sera and more recently, monoclonal antibodies) to prevent viral infection (e.g., rabies) and to treat active infection (e.g., coronavirus disease 2019 (COVID-19)).

The most extensive research on blocking viral entry into a cell has been done with Human Immunodeficiency Virus (HIV); two drugs that accomplish this are now approved by the Food and Drug Administration. None of the current agents that block the attachment and entry of an infectious agent are delivered topically, and none are known to be effective against coronavirus.

### SUMMARY

Some embodiments of the invention relate to a composition for the treatment of viral infections including a first active agent and a second active agent. In some embodiments, the first active agent can bind to an ACE2 receptor at a higher affinity than the virus causing the viral infection. In some embodiments, wherein the second active agent is a protease enzyme inhibitor.

In some embodiments, the protease enzyme inhibitor can be a TMPRRS2 protease inhibitor.

In some embodiments, the active agents can act synergistically.

In some embodiments, the ACE2 receptor blocker can be SSAA09E2 or nilotinib. In some embodiments, the protease enzyme inhibitor can be camostat.

In some embodiments, the virus can be SARS-CoV-2.

Some embodiments of the invention relates to a formulation including the composition and combined with a carrier suitable for administration to a patient in a topical/local manner.

In some embodiments, the formulation is the in form of a spray.

Some embodiments of the invention relate to a method for treating or preventing a viral infection in a patient. In some embodiments, the method can include administering the formulation to the patient.

In some embodiments, the first active agent does not bind to the virus.

In some embodiments, the treatment can be topical/local rather than systemic.

Some embodiments of the invention relate to a method for determining a composition for treatment or prophylaxis for a patient with a viral infection. In some embodiments, the method can include: identifying a virus that causes the viral infection; determining a receptor that the virus binds to, to cause the infection; determining a binding site on the receptor that the virus binds to; identifying a first active agent that has a higher binding affinity to the binding site than the virus and does not bind to the virus; optionally identifying a second active agent capable of acting synergistically with the first active agent, as measured by effectiveness in treating or preventing the viral infection; and/or identifying a dosing protocol for delivering the composition to the patient.

### BRIEF DESCRIPTION OF THE DRAWINGS

FIG. 1 is a molecular model depicting the interaction of the severe acute respiratory syndrome coronavirus 2 (SARS-CoV-2) Spike receptor binding domain (RBD) with the N-terminal binding pocket of the human angiotensin converting enzyme 2 (ACE2) molecule. The agent SSAA09E2 can bind with the ACE2 receptor in the area where the RBD normally seeks to attach. Another known ACE2 inhibitor, MLN-4760, binds to ACE2 at another site.

FIG. 2 depicts the effect of increasing concentrations of SSAA09E2 on the attachment and entry of the pseudovirus carrying the Spike protein of SARS-CoV-2 into the human cells expressing ACE2. A dose-dependent inhibition of pseudovirus infection was observed.

FIG. 3 depicts the effect of increasing concentrations of MNL-4760 on the attachment and entry of the pseudovirus carrying the S-protein of SARS-CoV-2 into the human cells expressing ACE2. No inhibition of viral infection was observed.

### DETAILED DESCRIPTION

The invention disclosed herein relates to a new therapeutic approach for the treatment or prevention of respiratory viruses like a coronavirus disease. The coronavirus disease can be caused by severe acute respiratory syndrome coronavirus 1 (SARS-CoV-1), severe acute respiratory syndrome coronavirus 2 (SARS-CoV-2), Middle East respiratory syndrome coronavirus (MERS-CoV), human coronavirus NL63 (HCoV-NL63), or the like. The respiratory virus can be any virus that utilizes the ACE2 protein to infect a cell.

The vast majority of pathogenic respiratory viruses that infect humans are transmitted from an infected person to a susceptible person through the air (e.g., cough, sneeze, vocalizing). In the case of SARS-CoV-2, the virus finds a cell in the upper airway that has a surface protein (the receptor) that is a match for a binding protein on the surface of the virus. After binding, a complex but rapid set of changes occurs that results in the virus entering the target cell. Once inside the target cell, the virus rapidly co-opts the cell's biological machinery to make more virus particles, which are quickly released and infect many more local cells. Once the infection is established, the person develops, for example, a common cold, COVID-19, or influenza (depending on the virus).

Some aspects of the invention relate to SARS-CoV-2 specifically binding to the ACE2 protein to infect a cell. ACE2 is a protein that is found on many cells in the human body but the most important are the superficial lining cells of the nose, throat, bronchial tubes, and lung tissue. In addition to the ACE2 receptor, SARS-CoV-2 requires the action of a second protein on the surface of the target cell in order be successful in the infection process. The TMPRRS2 protein is a transmembrane serine protease on the target cell that performs the critical and essential function of “priming” the virus S1 protein in order to facilitate a secure and stable binding of the virus to the receptor structure. Thus, embodiments of the invention relate to methods of delivering infectious agent receptor blockers to airway surface of ciliated respiratory epithelial cells that express ACE2 and TMPRRS2. The method of delivery can be by a topical nasal spray or other mechanisms of delivering an inhaled medication.

### Compositions

Compositions including one or more active agent(s) for the treatment of respiratory viral infections are provided. In some embodiments, the active agent is capable of attaching to one or more cellular surface receptor molecules or enzymes of respiratory or enteric tract lining cells. The active agent can be a chemical or biological agent. The one or more cellular surface receptor molecules or enzymes can be a molecule that is also used by an infectious agent to enter, infect, or damage a cell. The infectious agent can be a specific virus or any other infectious pathogen. The one or more chemical or biologic agent(s) can inhibit the ability of the infectious agent to attach to, colonize, infect, and/or damage the cell.

In some embodiments, the composition includes an active agent that is capable of binding to the ACE2 protein to inhibit the ability of the infectious agent to attach to, colonize, infect, and/or damage the cell. Thus, the composition can be used to treat or prevent human infection with any infectious agent that utilizes the ACE2 protein to infect and/or damage a cell. Known infectious agents that utilize ACE2 protein and thus can be included in the composition include, but are not limited to, coronaviruses such as SARS-CoV-1, SARS-CoV-2, MERS-CoV, and HCoV-NL63.

Some specific embodiments of the invention relate to the prevention and/or treatment of human infection with SARS-CoV-2. In some embodiments, the active agent is capable of binding to or otherwise blocking the ACE2 protein in the ACE2 SARS-CoV-2 binding pocket, which binding pocket is utilized by the receptor binding protein (RBP) localized on the SARS-CoV-2 S1 (Spike) protein. The binding of the active agent in the binding pocket is strong enough to block, destabilize, interfere, or disrupt the binding of the SARS-CoV-2 RBP to ACE2 and prevent infection of the cell that carries the ACE2 receptor.

In some embodiments, the active agent that binds to ACE2 protein can be a known ACE2 protein inhibitor or blocker that binds to the RBP. For example, the active agent can be a specific peptidomimetic small molecule such as SSAA09E2, nilotinib, or the like. In other embodiments, the active agent can be any synthetic agent that binds to the cell receptor of a pathogenic respiratory virus, including but not limited to SARS-CoV-2, with a binding affinity sufficient to interfere with virus binding and/or function. For example, the binding affinity can be about 0.1-100 uM. For example, the binding affinity can be about 1-10 uM. For example, the binding affinity can be about 0.1, 0.5, 1, 1.5, 2, 2.5, 3, 3.5, 4, 4.5, 5, 5.5, 6, 6.5, 7, 7.5, 8, 8.5, 9, 9.5, 10, 25, 50, 75, or 100 uM.

In some embodiments, the composition can include a second active agent. The second active agent can be a protease inhibitor that can bind to and inhibit the function of the TMPRRS2 serine protease enzyme. This enzyme is found on the surface of cells lining the respiratory tract and is important in cutting the SARS-CoV-2 RBP in a specific place that alters its conformation and stabilizes the molecular bond to the ACE2 receptor. This increases the efficiency of viral entry into the target respiratory epithelial cell. An example of a TMPRRS2 inhibitor includes but is not limited to camostat.

In some embodiments, the composition includes at least two active agents, which can act synergistically. In some embodiments, the composition can include an ACE2 protein receptor blocker and a TMPRRS2 protease enzyme inhibitor that act synergistically to prevent and/or treat infection with SARS-CoV-2.

The ratio of the synergistic ingredients can be about 1:1, 1:2, 1:3, 1:6, 1:9, 1:12, 2:1, 2:3, 2:9, 3:1, 3:2, 3:12, 6:1, 9:1, 9:2, 9:12, 12:1, 12:3, 12:9 or more or less.

Table 1 lists major ratios of pairwise combinations of synergistic active agents of the invention, named as ratios A through S where the first active agent is S1 and the second active agent is S2. For example, 51 can be an ACE2 protein receptor blocker and S2 can be a TMPRR2 protease enzyme inhibitor. While the table provides a range of ratios that can be useful, it is within the scope of the invention to provide formulations in which synergistic ratios are adapted to particular uses.

TABLE 1

| | Active Agent 2 | | | | | | | | | | | |
|---|---|---|---|---|---|---|---|---|---|---|---|---|
| Active | 1 part | | 2 parts | | 3 parts | | 6 parts | | 9 parts | | 12 parts | |
| Agent 1 | S2 | | S2 | | S2 | | S2 | | S2 | | S2 | |
| 1 part | 1:1 | (A) | 1:2 | (G) | 1:3 | (J) | 1:6 | (M) | 1:9 | (N) | 1:12 | (Q) |
| S1 | | | | | | | | | | | | |
| 2 parts | 2:1 | (B) | 2:2 | (A) | 2:3 | (K) | 2:6 | (J) | 2:9 | (O) | 2:12 | (M) |
| S1 | | | | | | | | | | | | |
| 3 parts | 3:1 | (C) | 3:2 | (H) | 3:3 | (A) | 3:6 | (G) | 3:9 | (J) | 3:12 | (R) |
| S1 | | | | | | | | | | | | |
| 6 parts | 6:1 | (D) | 6:2 | (C) | 6:3 | (B) | 6:6 | (A) | 6:9 | (K) | 6:12 | (G) |
| S1 | | | | | | | | | | | | |
| 9 parts | 9:1 | (E) | 9:2 | (I) | 9:3 | (C) | 9:6 | (H) | 9:9 | (A) | 9:12 | (S) |
| S1 | | | | | | | | | | | | |
| 12 parts | 12:1 | (F) | 12:2 | (D) | 12:3 | (L) | 12:6 | (B) | 12:9 | (P) | 12:12 | (A) |
| S1 | | | | | | | | | | | | |

Further information can be found in Kawase M, Shirato K, van der Hoek L, Taguchi F, Matsuyama S. Simultaneous treatment of human bronchial epithelial cells with serine and cysteine protease inhibitors prevents severe acute respiratory syndrome coronavirus entry. J Virol. 2012; 86(12):6537-6545. doi:10.1128/JVI.00094-12; and Hui K P Y, Cheung M C, Perera R A P M, et al. Tropism, replication competence, and innate immune responses of the coronavirus SARS-CoV-2 in human respiratory tract and conjunctiva: an analysis in ex-vivo and in-vitro cultures [published online ahead of print, 2020 May 7]. Lancet Respir Med. 2020; 8(7):687-695. doi:10.1016/52213-2600(20)30193-4; and Razizadeh M, Nikfar M, and Jui Y. Small molecules to Destabilize the ACE2RBD Complex: A Molecular Dynamics Study for Potential COVID-19 Therapeutics. ChemRxiv 2020 Dec. 6; doi: 10.26434/chemrxiv.13377119. preprint before publication. Each of the foregoing is fully incorporated by reference herein.

In some embodiments, the agents can be delivered or applied topically or by inhalation and/or otherwise applied superficially in a manner that does not produce clinically significant systemic levels of the agent(s).

Some embodiments of the invention relate to a formulation comprising the composition of active agents. In some embodiments, the formulation is in the form of a topical spray, nasal rinse, inhaled aerosol, or the like. The physiochemical characteristics of each identified active ingredient can be identified and the necessary excipients, solutions, stabilizers, and concentrations necessary to produce an effective topical product are defined as standard in the art so that the agent(s) can be delivered to the mucosal membranes of the upper and lower airway.

### Methods of Treatment

Some embodiments of the invention relate to administering the formulation topically to a patient diagnosed with or suspected of having COVID-19. In other embodiments the formulation can be used prophylactically to reduce a risk of infection with SARS-CoV-2, for example, in a person who was recently exposed to another individual with active COVID-19 infection.

For all embodiments of the invention, the active agent(s) can work by a proposed mechanism that interferes with an infectious virus at pre-symptomatic or early symptomatic stages of infection. This is achieved by the agent being delivered to the mucous membranes of the upper and/or lower airway as soon as possible after exposure to infection or at the earliest signs of illness. As a non-limiting example, the agent can be delivered up to about 24 hours after exposure to effectively prevent infection and up to about 48 hours after first symptoms to reduce the severity and duration of clinical illness. For example, the agent can be delivered up to about 4, 5, 6, 7, 8, 9, 10, 11, 12, 13, 14, 15, 16, 17, 18, 19, 20, 21, 22, 23, 24 hours or more after exposure to infection. For example, the agent can be delivered up to about 20, 21, 22, 23, 24, 25, 26, 27, 28, 29, 30, 31, 32, 33, 34, 35, 36, 37, 38, 39, 40, 41, 42, 43, 44, 45, 46, 47, 48 or more hours after first symptoms. The duration of treatment can be significantly shorter than would be required once infection is well established. The dosing interval can be determined primarily by the stability of the binding of each specific agent to the host cell receptor. A non-limiting example is the binding of SSAA09E2 to the ACE2 receptor where the estimated detachment half-life is approximately 2-3 hours and the concentrations of agent delivered to the upper airways by topical spray or mist to the upper can be 100-1000 times those necessary to inhibit the virus-receptor interaction (Ki>10 uM). In this non-limiting example the repeat dosing required can be every 4-6 hours for an agent such as SSAA09E2 to be effective in preventing or treating COVID-19 infection.

### Methods of Determining Active Agents for the Treatment of Viral Infections

The invention also encompasses methods for determining effective active agents. In some embodiments, the invention relates to a method for determining a composition for treatment or prophylaxis for a patient with a viral infection. The method can include identifying a virus that causes the viral infection; determining a receptor that the virus binds to, to cause the infection; determining a binding site on the receptor that the virus binds to; identifying a first active agent that has a higher binding affinity to the binding site than the virus and does not bind to the virus; optionally identifying a second active agent capable of acting synergistically with the first active agent, as measured by effectiveness in treating or preventing the viral infection; and identifying a dosing protocol for delivering the composition to the patient.

In some embodiments, the viral infection is infection of a coronavirus and the binding site is RBP.

### EXAMPLES

### Example 1

### Example Formulation

A formulation including an ACE2 receptor blocker (SSAA09E2) and a TMPRRS2 protease enzyme inhibitor (camostat) is delivered by topical spray to a patient. The topical spray contacts the nasopharynx and throat. In addition, or as an alternative, the formulation is delivered by inhaled mist or another respiratory delivery system to the bronchus and lungs. The patient experiences at least one of
(a) blocking of the infectious process and avoidance of any disease symptoms; and/or
(b) interference with the infectious process and
   (i) reduction of any disease symptoms and/or
   (ii) an accelerated recovery from any disease symptoms.

### Example 2

### Demonstration of Synergy

Experiments are done to demonstrate synergy of the active agents. Quantification of putative synergistic effects is based upon an adaptation of the Colby method (S. R. Colby, “Calculating Synergistic and Antagonistic Response of Herbicide Combinations.” Weeds 15(1): 20-23, 1967). Specifically, each putative synergistic agent is administered to cells in an in vitro model and/or to animals in an animal model of the infectious process, and the quantitative response is recorded. Then the combination of the active agents is made at 3:1, 1:1, and 1:3 ratios and each combination is contacted with the model assay system along a dilution gradient. The response of each treatment is quantified and a calculation of synergy is derived. Synergistic combinations in terms of ratios of the two active agents and in terms of dose-response are further optimized in subsequent tests based upon the first series of tests. Accordingly, a synergistic formulation and dosing protocol is developed.

### Example 3

### Treatment

Patients diagnosed with COVID-19 are treated with a synergistic formulation including an ACE2 receptor blocker (SSAA09E2) and a TMPRRS2 protease enzyme inhibitor (camostat) that is delivered by topical spray to the nasopharynx and throat, and/or by inhaled mist or another respiratory delivery system to the bronchus and lungs. After treatment, patients demonstrate improved symptoms related to COVID-19.

### Example 4

### Binding of ACE2 Receptor Blocker Assay

This example provides a method to quantitate the ability of a specific agent to block the interaction of the SARS-CoV-2-RBP with the human ACE2 receptor. An assay with 293T cells stably expressing hACE2 (293T-hACE2) with a lentiviral vector is used. Expression of hACE2 in the transduced cells is confirmed by western blotting. A pseudotyped SARS-CoV-2 is created using Fluc-packaged HIV-based lentiviral particles containing the S protein of SARS-COV-2. The 293T-hACE2 cells are exposed to concentrations gradients of candidate receptor blocking agents, or combinations of agents. The assay is used to quantify the reduction of the infectivity of the pseudovirus carrying the SARS-CoV-2 S1 (Spike) protein. Agents that produce low infectivity scores are selected as potential agents of interest for further laboratory testing and subsequent studies in animal models of COVID-19 infection.

### Example 4

### SSAA09E2 as an Agent

Effective and efficient in vitro screening is critical for candidate compounds identified as having the molecular and structural attributes needed to inhibit viral binding and entry into the host cell. One of these screening methods was selected as a high-throughput process to confirm the necessary mechanism of action of a specific candidate agent, SSAA09E2, in a laboratory study relevant to COVID-19 infection. Coronaviruses get the name from the “corona” or halo seen around the virus particles in an electron microscope. The halo is from large structural proteins (e.g., S-protein, also known as the “Spike protein”) which protrude out of the virus. Viral entry into human cells is initiated when the Spike protein binds to the human cell's surface protein called ACE2. The virus cannot infect cells that lack ACE2 on the surface. Therefore, blocking the Spike-ACE2 interaction is a potential therapeutic strategy.

A study was devised to evaluate whether a specific agent that binds to the ACE2 at the receptor binding pocket could reduce SARS COVID to entry into a human cell. The comparison agent is the ACE2 inhibitor which binds to the ACE2protein at another site on the molecule, inhibiting the enzyme activity of the protein but not blocking the binding of the virus.

Because SARS-CoV-2 coronavirus is the infectious agent that causes the COVID-19, it is hazardous to study it in infectious form; a pseudovirus is a safer alternative. Pseudoviruses are replication incompetent virus-like-particles based on HIV, where the HIV genome has been replaced with a single reporter luciferase gene. Upon infection by the pseudovirus and release of the reporter gene, the luciferase enzyme is produced. This enzyme drives a bioluminescent reaction that can be detected and quantified. Three pseudoviruses were engineered; two are internal positive and negative controls. One was coated with the SARS-CoV-2 Spike protein. This pseudovirus will only infect cells that have an abundance of ACE2 on the surface; for this reason, the human cells target cells were engineered to display an abundance of ACE2 on their cell surface.

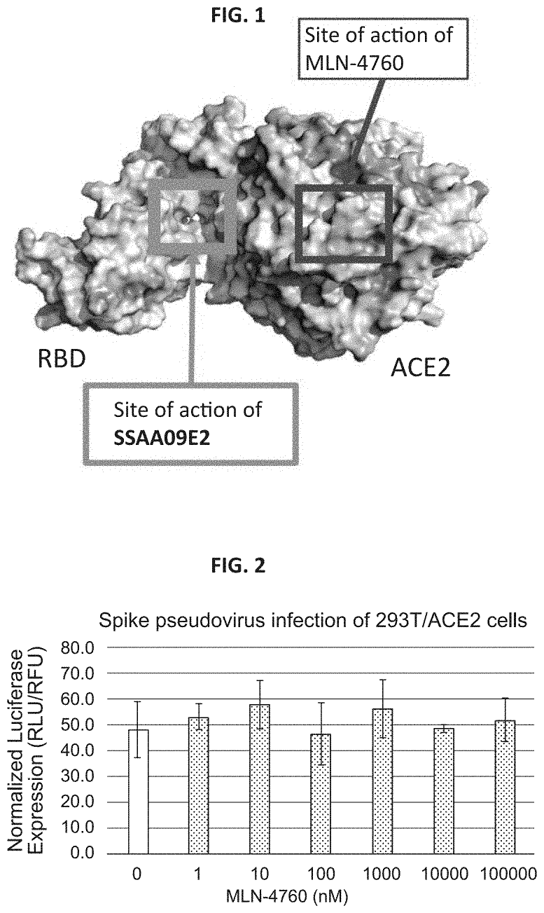

The agents SSAA09E2 and MNL-4760 were selected (see FIG. 1).

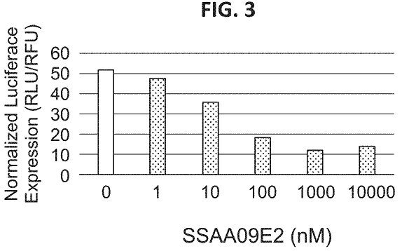

MLN-4760 was considered to be a useful comparator agent serving as a “functional negative control” in the study. The two agents were tested at increasing concentrations. MLN-4760 showed no ability to block the entrance to the pseudovirus (see FIG. 2). However, the initial data indicate that SSAA09E2 was effective at blocking infection in concentrations that would be easily achieved with topical administration to the upper airway mucosa (see FIG. 3).

This example shows that SSAA09E2 is a viable agent as a topical inhibitor of SARS-CoV-2. It also demonstrates the existence of a robust technical method to identify agents that would be effective against coronavirus infections and, with appropriate modifications, to identify attachment inhibitors that block other respiratory viruses.

The various methods and techniques described above provide a number of ways to carry out the application. Of course, it is to be understood that not necessarily all objectives or advantages described are achieved in accordance with any particular embodiment described herein. Thus, for example, those skilled in the art will recognize that the methods can be performed in a manner that achieves or optimizes one advantage or group of advantages as taught herein without necessarily achieving other objectives or advantages as taught or suggested herein. A variety of alternatives are mentioned herein. It is to be understood that some embodiments specifically include one, another, or several features, while others specifically exclude one, another, or several features, while still others mitigate a particular feature by including one, another, or several other features.

Furthermore, the skilled artisan will recognize the applicability of various features from different embodiments. Similarly, the various elements, features and steps discussed above, as well as other known equivalents for each such element, feature or step, can be employed in various combinations by one of ordinary skill in this art to perform methods in accordance with the principles described herein. Among the various elements, features, and steps some will be specifically included and others specifically excluded in diverse embodiments.

Although the application has been disclosed in the context of certain embodiments and examples, it will be understood by those skilled in the art that the embodiments of the application extend beyond the specifically disclosed embodiments to other alternative embodiments and/or uses and modifications and equivalents thereof.

In some embodiments, any numbers expressing quantities of ingredients, properties such as molecular weight, reaction conditions, and so forth, used to describe and claim certain embodiments of the disclosure are to be understood as being modified in some instances by the term “about.” Accordingly, in some embodiments, the numerical parameters set forth in the written description and any included claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the application are approximations, the numerical values set forth in the specific examples are usually reported as precisely as practicable.

In some embodiments, the terms “a” and “an” and “the” and similar references used in the context of describing a particular embodiment of the application (especially in the context of certain claims) are construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (for example, “such as”) provided with respect to certain embodiments herein is intended merely to better illuminate the application and does not pose a limitation on the scope of the application otherwise claimed. No language in the specification should be construed as indicating any non-claimed element essential to the practice of the application.

Variations on preferred embodiments will become apparent to those of ordinary skill in the art upon reading the foregoing description. It is contemplated that skilled artisans can employ such variations as appropriate, and the application can be practiced otherwise than specifically described herein. Accordingly, many embodiments of this application include all modifications and equivalents of the subject matter recited in the claims appended hereto as permitted by applicable law. Moreover, any combination of the above-described elements in all possible variations thereof is encompassed by the application unless otherwise indicated herein or otherwise clearly contradicted by context.

All patents, patent applications, publications of patent applications, and other material, such as articles, books, specifications, publications, documents, things, and/or the like, referenced herein are hereby incorporated herein by this reference in their entirety for all purposes, excepting any prosecution file history associated with same, any of same that is inconsistent with or in conflict with the present document, or any of same that may have a limiting effect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

In closing, it is to be understood that the embodiments of the application disclosed herein are illustrative of the principles of the embodiments of the application. Other modifications that can be employed can be within the scope of the application. Thus, by way of example, but not of limitation, alternative configurations of the embodiments of the application can be utilized in accordance with the teachings herein. Accordingly, embodiments of the present application are not limited to that precisely as shown and described.

What is claimed is:

1. A composition for the treatment of viral infections comprising a first active agent and a second active agent, wherein the first active agent binds to an ACE2 receptor at a higher affinity than a virus causing the viral infection, wherein the second active agent is a protease enzyme inhibitor.

2. The composition of claim 1, wherein the protease enzyme inhibitor is a TMPRRS2 protease inhibitor.

3. The composition of claim 1, wherein the active agents act synergistically.

4. The composition of claim 1, wherein the ACE2 receptor blocker is SSAA09E2 or nilotinib and wherein the protease enzyme inhibitor is camostat.

5. The composition of claim 1, wherein the virus is SARS-CoV-2.

6. A formulation comprising the composition of claim 1, combined with a carrier suitable for administration to a patient in a topical/local manner.

7. The formation of claim 6, wherein the formulation is the in form of a spray.

8. A method for treating or preventing a viral infection in a patient comprising administering a formulation comprising the composition of claim 1 to the patient.

9. The method of claim 8, wherein the first active agent does not bind to the virus.

10. The method of claim 8, wherein the treatment is topical/local rather than systemic.

11. A method for determining a composition for treatment or prophylaxis for a patient with a viral infection comprising:
a. identifying a virus that causes the viral infection;
b. determining a receptor that the virus binds to, to cause the infection;
c. determining a binding site on the receptor that the virus binds to;
d. identifying a first active agent that has a higher binding affinity to the binding site than the virus and does not bind to the virus;
e. optionally identifying a second active agent capable of acting synergistically with the first active agent, as measured by effectiveness in treating or preventing the viral infection; and
f. identifying a dosing protocol for delivering the composition to the patient.
